(19) United States
(12) Patent Application Publication
(10) Pub. No.: US 2006/0021155 A1
(43) Pub. Date: Feb. 2, 2006

(54) Dyeing composition containing a laccase and method for dyeing keratinous fibres

(75) Inventors: Gerard Lang, Saint Prix (FR); Jean Cotteret, Verneuil/Scine (FR)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: L'Oreal S.A

(21) Appl. No.: 10/992,474

(22) Filed: Nov. 19, 2004

Related U.S. Application Data
(63) Continuation of application No. 09/600,132, filed on Aug. 14, 2000, now abandoned.

(30) Foreign Application Priority Data
Jan. 13, 1998 (FR) 98 00250

(51) Int. Cl. A61K 8/00

(52) U.S. Cl. 8/405

(57) ABSTRACT

The invention concerns a cosmetic composition for oxidation dyeing of keratinous fibres comprising in a support suitable for keratinous fibre dyeing: (a) at least an enzyme such as laccase; (b) at least a particular alkalising agent; (c) at least an oxidation colouring agent, as well as the dyeing methods using said composition.

The present invention relates to a composition for the oxidation dyeing of keratinous fibres comprising at least one enzyme of the laccase type, at least one oxidation dye and at least one particular alkalinizing agent as well as the methods of dyeing keratinous fibres, in particular human hair using this composition.

It is known to dye keratinous fibres, and in particular human hair, with dyeing compositions containing oxidation dye precursors, in particular ortho- and para-phenylenediamines, ortho- or para-aminophenols, heterocyclic bases generally called oxidation bases. The oxidation dye precursors, or oxidation bases, are colourless or weakly coloured compounds which, combined with oxidizing products, can give rise to coloured compounds by a process of oxidative condensation.

It is also known that the shades obtained with these oxidation bases can be varied by combining them with couplers or colour modifiers, the latter being chosen in particular from aromatic meta-diamines, meta-aminophenols, meta-diphenols and certain heterocyclic compounds.

The variety of molecules used in oxidation bases and couplers allows a rich palette of colours to be obtained.

The so-called “permanent” colour obtained by means of these oxidation dyes should moreover satisfy a number of requirements. Thus, it should have no drawbacks from the toxicological point of view, it should make it possible to obtain shades of the desired intensity and it should exhibit good resistance towards external agents (light, adverse weather conditions, washing, permanent waving, perspiration, rubbing).

The dyes should also make it possible to cover grey hair, and thus should be the least selective possible, that is to say they should make it possible to obtain the smallest possible differences in colour all along the same keratinous fibre, which may indeed be differently sensitized (i.e. damaged) between its tip and its root.

The oxidation dyeing of keratinous fibres is generally carried out in an alkaline medium, in the presence of hydrogen peroxide. However, the use of alkaline media in the presence of hydrogen peroxide has the disadvantage of causing substantial degradation of the fibres, as well as decolouring of the keratinous fibres which is not always desirable.

The oxidation dyeing of keratinous fibres can also be carried out with the aid of oxidizing systems different from hydrogen peroxide such as enzymatic systems. Thus, it has already been proposed in Patent U.S. Pat. No. 3,251,742, Patent Applications FR-A-2,112,549, FR-A-2,694,018, EP-A-0,504,005, WO95/07988, WO95/33836, WO95/33837, WO96/00290, WO97/19998 and WO97/19999 to dye keratinous fibres with compositions comprising at least one oxidation dye in combination with enzymes of the laccase type, the said compositions being brought into contact with atmospheric oxygen. These dyeing formulations, although used under conditions which do not cause degradation of the keratinous fibres comparable to that caused by dyeings carried out in the presence of hydrogen peroxide, lead to colours which are still inadequate both from the point of view of homogeneity of the:colour distributed along the fibre (“unison”), from the point of view of chromaticity (luminosity) and of the dyeing power.

The aim of the present invention is to solve the problems mentioned above.

The applicant has surprisingly discovered novel compositions containing, as oxidizing system, at least one enzyme of the laccase type and at least one particular alkalinizing agent which will be defined in greater detail below, which may constitute, in the presence of oxidation dyes, ready-to-use dyeing formulations leading to colours which are more homogeneous, more intense and more chromatic without causing significant degradation or decolouring of the keratinous fibres, exhibiting low selectivity and good resistance to various attacks to which the hair may be subjected.

These discoveries form the basis of the present invention.

The first subject of the present invention is therefore a ready-to-use composition intended for the oxidation dyeing of keratinous fibres, in particular human keratinous fibres and more particularly human hair, comprising, in a carrier appropriate for dyeing keratinous fibres:
(a) at least one enzyme of the laccase type;
(b) at least one alkalinizing agent chosen from the group consisting of:

(i) a basic amino acid;

(ii) a compound of the following formula (A): X(OH)ₙ in which X represents K, Li when n=1; X represents Mg, Ca when n=2; X represents N⁺R₁R₂R₃R₄ with R₁,R₂,R₃,R₄, which are identical or different, denoting a C₁-C₄ alkyl radical, a C₁-C₄ monohydroxyalkyl or C₂-C₄ polyhydroxyalkyl radical, when n=1;

(iii) a compound of the following formula (B): 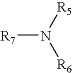
in which R₅ denotes a C₁-C₆ alkyl radical, a C₁-C₆ monohydroxyalkyl or C₂-C₆ polyhydroxyalkyl radical; R₆, R₇, which are identical or different, denote a hydrogen atom, a C₁-C₆ alkyl radical, a C₁-C₆ monohydroxyalkyl or C₂-C₆ polyhydroxyalkyl radical;
with the proviso that
R₅,R₆,R₇ do not simultaneously denote the C₂ β-hydroxy-alkyl radical,
if R₆ and R₇ simultaneously denote H, then R₅ does not denote a C₂ monohydroxyalkyl or branched C₄ monohydroxyalkyl radical,
if R₅ denotes hydrogen or a C₁-C₆ alkyl radical and at the same time R₆ denotes a C₁-C₆ alkyl radical, then R₇ does not denote H or a C₁-C₆ alkyl radical;

(iv) a compound of the following formula (C): 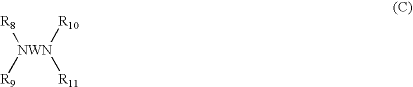
in which W is a propylene residue optionally substituted with a hydroxyl group or a C₁-C₄ alkyl radical; R₈, R₉, R₁₀ and R₁₁, which are identical or different, represent a hydrogen atom, a C₁-C₄ alkyl or C₁-C₄ hydroxyalkyl radical;
(c) at least one oxidation dye with the exception of autooxidizable indole dyes.

The laccase(s) used in the ready-to-use dye composition in accordance with the invention may be chosen in particular from laccases of plant origin, animal origin, fungal origin (yeasts, moulds, fungi) or bacterial origin, organisms which may be of mono- or pluricellular origin. They can be obtained by biotechnology.

Among the laccases of plant origin which can be used according to the invention, there may be mentioned the laccases produced by plants which perform chlorophyll synthesis as indicated in Application FR-A-2,694,018 such as those found in the extracts of Anacardiaceae such as for example the extracts of Magnifera indica, Schinus molle or Pleiogynium timoriense, in the extracts of Podocarpaceae, Rosmarinus off., Solanum tuberosum, Iris sp., Coffea sp., Daucus carrota, Vinca minor, Persea americana, Catharenthus roseus, Musa sp., Malus pumila, Gingko biloba, Monotropa hypopithys (Indian pipe), Aesculus sp., Acer pseudoplatanus, Prunus persica, Pistacia palaestina.

Among the laccases of fungal origin optionally obtained by biotechnology which can be used according to the invention, there may be mentioned the laccase(s) derived from Polyporus versicolor, Rhizoctonia practicola and Rhus vernicifera as indicated in Applications FR-A-2,112,549 and EP-A-504005, those described in Patent Application WO95/07988, WO95/33836, WO95/33837, WO96/00290, WO97/19998 and WO97/19999, whose content is an integral part of the present description, such as for example those derived from Scytalidium, Polyporus pinsitus, Myceliophtora thermophila, Rhizoctonia solani, Pyricularia orizae, or variants thereof. There may also be mentioned those derived from Tramates versicolor, Fomes fomentarius, Chaetomium thermophile, Neurospora crassa, Coriolus versicol, Botrytis cinerea, Rigidoporus lignosus, Phellinus noxius, Pleurotus ostreatus, Aspergillus nidulans, Podospora anserina, Agaricus bisporus, Ganoderma lucidum, Glomerella cingulata, Lactarius piperatus, Russula delica, Heterobasidion annosum, Thelephora terrestris, Cladosporium cladosporiodes, Cerrena unicolor, Coriolus hirsutus, Ceriporiopsis subvermispora, Coprinus cinereus, Panaeolus papilionaceus, Panaeolus sphinctrinus, Schizophyllum commune, Dichomitius squalens and variants thereof.

The laccases of fungal origin optionally obtained by biotechnology will be preferably chosen.

The enzymatic activity of the laccases of the invention which have syringaldazine among their substrates can be defined from the oxidation of syringaldazine under aerobic conditions. The lacu unit corresponds to the quantity of enzyme catalysing the conversion of 1 mmol of syringaldazine per minute at pH 5.5 at 30° C. The unit u corresponds to the quantity of enzyme producing a delta absorbance at 530 nm of 0.001 per minute using syringaldazine as substrate, at 30° C. and at pH 6.5.

The enzymatic activity of the laccases of the invention can also be defined from the oxidation of para-phenylenediamine. The lacu unit corresponds to the quantity of enzyme producing a delta absorbance at 496.5 nm of 0.001 per minute using para-phenylenediamine as substrate (64 mM) at 30° C. and at pH 5. According to the invention, it is preferable to determine the enzymatic activity in lacu units.

The quantities of laccase used in the compositions of the invention will vary according to the nature of the laccase chosen. Preferably, they will vary from 0.5 to 2000 lacu, or from 1000 to 4×10⁷ u units, or from 20 to 2×10⁶ lacu units per 100 g of composition.

For the purposes of the present invention, i.e. in the preceding text and in the text which follows, “basic amino acid” defines either (i) an amino acid having, in addition to the amine function positioned in α with respect to the carboxyl group, an additional cationic (or basic) group; or (ii) an amino acid having a cationic (or basic) (hydrophilic) side chain; or (iii) an amino acid carrying a side chain consisting of a nitrogenous base. These definitions are generally known and are published in general biochemistry books such as J. H. WEIL (1983) pages 5 and the following pages, Lubert STRYER (1995) page 22, A. LEHNINGER (1993) pages 115-116, DE BOECK-WESMAEL (1994) pages 57-59.

The basic amino acids in accordance with the invention are preferably chosen from those corresponding to the following formula (D): 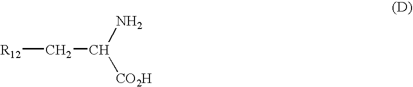
where R₁₂ denotes a group chosen from:

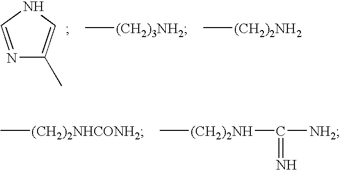

The compounds corresponding to formula (D) are histidine, lysine, ornithine, citrulline, arginine.

In the compounds of formula (A) or (B), according to the invention, the alkyl radicals may be linear or branched and the polyhydroxyalkyl radicals denote radicals comprising from 2 to 6 hydroxyl groups and preferably from 2 to 4.

The compounds of formula (B), according to the invention, are preferably chosen from the group consisting of diethanolamine, monoisopropanolamine, diisopropanolamine, triisopropanolamine, 2-amino-2-methyl-1,3-propanediol, 2-amino-2-ethyl-1,3-propane-diol, 2-amino-1-n-butanol, 1-diethylamino-2,3-propane-diol, tris(hydroxymethyl)aminomethane, ethylmono-ethanolamine.

The compositions in accordance with the invention contain the particular alkalinizing agents defined above in contents by weight which may range from 0.001% to 20%, preferably from 0.01% to 5% and still more preferably from 0.05% to 3%, relative to the total weight of the composition.

The nature of the oxidation dye(s) used in the ready-to-use dyeing composition is not critical. They are chosen from oxidation bases and/or couplers.

The oxidation bases may be chosen in particular from para-phenylenediamines, double bases, para-aminophenols, ortho-aminophenols and heterocyclic oxidation bases.

Among the para-phenylenediamines which can be used as oxidation base in the dyeing composition in accordance with the invention, there may be mentioned in particular the compounds of the following formula (I) and their addition salts with an acid: 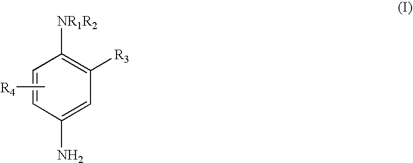
in which:
R₁ represents a hydrogen atom, a C₁-C₄ alkyl radical, a monohydroxy(C₁-C₄ alkyl) radical, a polyhydroxy-(C₂-C₄ alkyl) radical, a (C₁-C₄)alkoxy (C₁-C₄) alkyl radical, a C₁-C₄ alkyl radical substituted with a nitrogen-containing group, a phenyl radical or a 4′-aminophenyl radical;
R₂ represents a hydrogen atom, a C₁-C₄ alkyl radical, a monohydroxy(C₁-C₄ alkyl) radical, a polyhydroxy(C₂-C₄ alkyl) radical, a (C₁-C₄)alkoxy(C₁-C₄)alkyl radical or a C₁-C₄ alkyl radical substituted with a nitrogen-containing group;
R₃ represents a hydrogen atom, a halogen atom such as a chlorine, bromine, iodine or fluorine atom, a C₁-C₄ alkyl radical, a monohydroxy(C₁-C₄ alkyl) radical, a hydroxy(C₁-C₄ alkoxy) radical, an acetylamino(C₁-C₄ alkoxy) radical, a mesylamino(C₁-C₄ alkoxy) radical or a carbamoylamino(C₁-C₄ alkoxy) radical,
R₄ represents a hydrogen or halogen atom or a C₁-C₄ alkyl radical.

Among the nitrogen-containing groups of formula (I) above, there may be mentioned in particular the amino, mono(C₁-C₄)alkylamino, (C₁-C₄)dialkylamino, (C₁-C₄) trialkylamino, monohydroxy(C₁-C₄)alkylamino, imidazolinium and ammonium radicals.

Among the para-phenylenediamines of formula (I) above, there may be mentioned more particularly para-phenylenediamine, para-tolylenediamine, 2-chloro-para-phenylenediamine, 2,3-dimethyl-para-phenylene-diamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylene-diamine, N,N-dimethyl-3-methyl-para-phenylenediamine, N,N-(ethyl-β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, and their addition salts with an acid.

Among the para-phenylenediamines of formula (I) above, there are most particularly preferred para-phenylenediamine, para-tolylenediamine, 2-isopropyl-para-phenylenediamine, 2-β-hydroxyethyl-para-phenylenediamine, 2-β-hydroxyethyloxy-para-phenylene-diamine, 2,6-diethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine, 2-β-acetylaminoethyloxy-para-phenylenediamine, and their addition salts with an acid.

According to the invention, “double bases” is understood to mean the compounds containing at least two aromatic rings on which amino and/or hydroxyl groups are carried.

Among the double bases which can be used as oxidation bases in the dyeing compositions in accordance with the invention, there may be mentioned in particular the compounds corresponding to the following formula (II), and their addition salts with an acid: 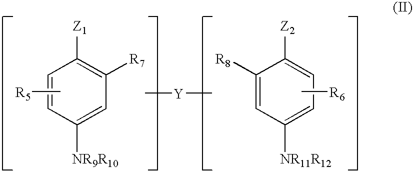
in which:
Z₁ and Z₂, which are identical or different, represent a hydroxyl or —NH₂ radical which may be substituted with a C₁-C₄ alkyl radical or with a linking arm Y;
the linking arm Y represents a linear or branched alkylene chain comprising from 1 to 14 carbon atoms, which may be interrupted by or which may end with one or more nitrogen-containing groups and/or one or more heteroatoms such as oxygen, sulphur or nitrogen atoms, and optionally substituted with one or more hydroxyl or C₁-C₆ alkoxy radicals;
R₅ and R₆ represent a hydrogen or halogen atom, a C₁-C₄ alkyl radical, a monohydroxy(C₁-C₄ alkyl) radical, a polyhydroxy(C₂-C₄ alkyl) radical, an amino(C₁-C₄ alkyl) radical or a linking arm Y;
R₇, R₈, R₉, R₁₀, R₁₁ and R₁₂, which are identical or different, represent a hydrogen atom, a linking arm Y or a C₁-C₄ alkyl radical;
   it being understood that the compounds of formula (II) contain only one linking arm Y per molecule.

Among the nitrogen-containing groups of formula (II) above, there may be mentioned in particular the amino, mono(C₁-C₄)alkylamino, (C₁-C₄) dialkylamino, (C₁-C₄) trialkylamino, monohydroxy(C₁-C₄)alkylamino, imidazolinium and ammonium radicals.

Among the double bases of formulae (II) above, there may be mentioned more particularly N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)-tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylene-diamine, 1,8-bis(2,5-diaminophenoxy)-3,5-dioxaoctane, and their addition salts with an acid.

Among these double bases of formula (II), N,N′-bis(β-hydroxyethyl)-N,N′-bis(4′-aminophenyl)-1,3-diaminopropanol, 1,8-bis(2,5-diaminophenoxy)-3,5-dioxaoctane or one of their addition salts with an acid are particularly preferred.

Among the para-aminophenols which can be used as oxidation bases in the dyeing compositions in accordance with the invention, there may be mentioned in particular the compounds corresponding to the following formula (III), and their addition salts with an acid: 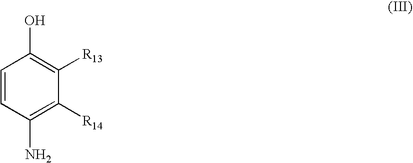
in which:
R₁₃ represents a hydrogen or halogen atom, a C₁-C₄ alkyl, monohydroxy(C₁-C₄ alkyl), (C₁-C₄)alkoxy(C₁-C₄)-alkyl, amino(C₁-C₄ alkyl) or hydroxy(C₁-C₄)alkylamino-(C₁-C₄ alkyl) radical,
R₁₄ represents a hydrogen or halogen atom, a C₁-C₄ alkyl, monohydroxy(C₁-C₄ alkyl), polyhydroxy(C₂-C₄ alkyl), amino(C₁-C₄ alkyl), cyano(C₁-C₄ alkyl) or (C₁-C₄)alkoxy(C₁-C₄)alkyl radical,
   it being understood that at least one of the radicals R₁₃ or R₁₄ represents a hydrogen atom.

Among the para-aminophenols of formula (III) above, there may be mentioned more particularly para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol, 4-amino-2-fluorophenol, and their addition salts with an acid.

Among the ortho-aminophenols which can be used as oxidation bases in the dyeing compositions in accordance with the invention, there may be mentioned more particularly 2-aminophenol, 2-amino-5-methylphenol, 2-amino-6-methylphenol, 5-acetamido-2-aminophenol, and their addition salts with an acid.

Among the heterocyclic bases which can be used as oxidation bases in the dyeing compositions in accordance with the invention, there may be mentioned more particularly pyridine derivatives, pyrimidine derivatives, pyrazole derivatives, pyrazolopyrimidine derivatives, and their addition salts with an acid.

Among the pyridine derivatives, there may be mentioned more particularly the compounds described for example in Patents GB 1,026,978 and GB 1,153,196, such as 2,5-diaminopyridine, 2-(4-methoxyphenyl)amino-3-aminopyridine, 2,3-diamino-6-methoxypyridine, 2-(β-methoxyethyl)amino-3-amino-6-methoxypyridine, 3,4-diaminopyridine, and their addition salts with an acid.

Among the pyrimidine derivatives, there may be mentioned more particularly the compounds described for example in German Patent DE 2,359,399 or Japanese Patents JP 88-169,571 and JP 91-333,495 or Patent Application WO 96/15765, such as 2,4,5,6-tetra-aminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine, and their addition salts with an acid.

Among the pyrazole derivatives, there may be mentioned more particularly the compounds described in Patents DE 3,843,892, DE 4,133,957 and Patent Applications WO 94/08969, WO 94/08970, FR-A-2,733,749 and DE 195 43 988 such as 4,5-diamino-1-methylpyrazole, 3,4-diaminopyrazole, 4,5-diamino-1-(4′-chlorobenzyl)-pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methyl-pyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropyl-pyrazole, 4-amino-5-(2′-aminoethyl)amino-1,3-dimethyl-pyrazole, 3,4,5-triaminopyrazole, 1-methyl-3,4,5-triaminopyrazole, 3,5-diamino-1-methyl-4-methylamino-pyrazole, 3,5-diamino-4-(β-hydroxyethyl)amino-1-methylpyrazole, and their addition salts with an acid.

Among the pyrazolopyrimidine derivatives, there may be mentioned more particularly the pyrazolo[1,5-a]pyrimidines of the following formula (IV), their addition salts with an acid or with a base and their tautomeric forms, when a tautometic equilibrium exists: 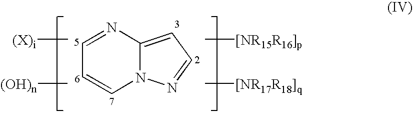
in which:
R₁₅, R₁₆, R₁₇ and R₁₈, which are identical or different, denote a hydrogen atom, a C₁-C₄ alkyl radical, an aryl radical, a C₁-C₄ hydroxyalkyl radical, a C₂-C₄ polyhydroxyalkyl radical, a (C₁-C₄)alkoxy(C₁-C₄ alkyl) radical, a C₁-C₄ aminoalkyl radical (it being possible for the amine to be protected with an acetyl, ureido or sulphonyl radical), a (C₁-C₄)alkylamino(C₁-C₄ alkyl) radical, a di-[(C₁-C₄)alkyl]amino(C₁-C₄ alkyl) radical (it being possible for the dialkyl radicals to form a carbon-containing ring or a 5- or 6-membered heterocycle), a hydroxy(C₁-C₄)alkyl- or di-[hydroxy(C₁-C₄)alkyl]-amino(C₁-C₄ alkyl) radical,
the X radicals, which are identical or different, denote a hydrogen atom, a C₁-C₄ alkyl radical, an aryl radical, a C₁-C₄ hydroxyalkyl radical, a C₂-C₄ polyhydroxyalkyl radical, a C₁-C₄ aminoalkyl radical, a (C₁-C₄)alkylamino(C₁-C₄ alkyl) radical, a di-[(C₁-C₄)alkyl]amino(C₁-C₄ alkyl) radical (it being possible for the dialkyls to form a carbon-containing ring or a 5- or 6-membered heterocycle), a hydroxy(C₁-C₄)alkyl or di-[hydroxy(C₁-C₄)alkyl]-amino(C₁-C₄ alkyl) radical, an amino radical, a (C₁-C₄)alkyl- or di-[(C₁-C₄)alkyl]-amino radical; a halogen atom, a carboxylic acid group, a sulphonic acid group;
i equals 0, 1, 2 or 3;
p equals 0 or 1;
q equals 0 or 1;
n equals 0 or 1;
   with the proviso that:
the sum p+q is different from 0;
when p+q is equal to 2, then n equals 0 and the groups NR₁₅R₁₆ and NR₁₇R₁₈ occupy positions (2,3); (5,6); (6,7); (3,5) or (3,7);
when p+q is equal to 1, then n equals 1 and the group NR₁₅R₁₆ (or NR₁₇R₁₈) and the OH group occupy positions (2,3); (5,6); (6,7); (3,5) or (3,7).

When the pyrazolo[1,5-a]pyrimidines of formula (IV) above are such that they comprise a hydroxyl group on one of the positions 2, 5 or 7 at the a position with respect to a nitrogen atom, a tautomeric equilibrium exists which is represented for example by the following scheme:

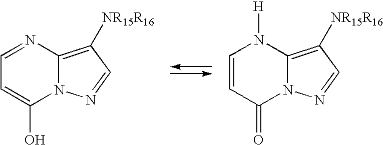

Among the pyrazolo[1,5-a]pyrimidines of formula (IV) above, there may be mentioned in particular:
pyrazolo[1,5-a]pyrimidine-3,7-diamine;
2,5-dimethylpyrazolo[1,5-a]pyrimidine-3,7-diamine;
pyrazolo[1,5-a]pyrimidine-3,5-diamine;
2,7-dimethylpyrazolo[1,5-a]pyrimidine-3,5-diamine;
3-aminopyrazolo[1,5-a]pyrimidin-7-ol;
3-aminopyrazolo[1,5-a]pyrimidin-5-ol;
2-(3-aminopyrazolo[1,5-a]pyrimidin-7-ylamino)ethanol;
2-(7-aminopyrazolo[1,5-a]pyrimidin-3-ylamino)ethanol;
2-[(3-aminopyrazolo[1,5-a]pyrimidin-7-yl)(2-hydroxy-ethyl)amino]ethanol;
2-[(7-aminopyrazolo[1,5-a]pyrimidin-3-yl)(2-hydroxy-ethyl)amino]ethanol;
5,6-dimethylpyrazolo[1,5-a]pyrimidine-3,7-diamine;
2,6-dimethylpyrazolo[1,5-a]pyrimidine-3,7-diamine;
2,5,N7,N7-tetramethylpyrazolo[1,5-a]pyrimidine-3,7-diamine;
   and their addition salts and their tautomeric forms, when a tautomeric equilibrium exists.

The pyrazolo[1,5-a]pyrimidines of formula (IV) above may be prepared by cyclization from an aminopyrazole according to the syntheses described in the following references:
EP 628559 BEIERSDORF-LILLY
R. Vishdu, H. Navedul, Indian J. Chem., 34b(6), 514, 1995.
N. S. Ibrahim, K. U. Sadek, F. A. Abdel-Al, Arch. Pharm., 320, 240, 1987.
R. H. Springer, M. B. Scholten, D. E. O'Brien,
T. Novinson, J. P. Miller, R. K. Robins, J. Med. Chem., 25, 235, 1982.
T. Novinson, R. K. Robins, T. R. Matthews, J. Med. Chem., 20, 296, 1977.
U.S. Pat. No. 3,907,799 ICN PHARMACEUTICALS

The pyrazolo[1,5-a]pyrimidines of formula (IV) above can also be prepared by cyclization from hydrazine according to the syntheses described in the following references:
A. McKillop and R. J. Kobilecki, Heterocycles, 6(9), 1355, 1977.
E. Alcade, J. De Mendoza, J. M. Marcia-Marquina, C. Almera, J. Elguero, J. Heterocyclic Chem., 11(3), 423, 1974.
K. Saito, I. Hori, M. Higarashi, H. Midorikawa, Bull. Chem. Soc. Japan, 47(2), 476, 1974.

The oxidation base(s) preferably represent from 0.0005 to 12% by weight approximately of the total weight of the dyeing composition in accordance with the invention, and still more preferably from 0.005 to 6% by weight approximately of this weight.

The coupler(s) which can be used in the ready-to-use dyeing composition in accordance with the invention are those conventionally used in oxidation dyeing compositions, that is to say meta-phenylene-diamines, meta-aminophenols, meta-diphenols, heterocyclic couplers, and their addition salts with an acid.

These couplers may be chosen in particular from 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxy-benzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, sesamol, α-naphthol, 6-hydroxyindole, 4-hydroxyindole, 4-hydroxy-N-methylindole, 6-hydroxyindoline, 2,6-dihydroxy-4-methylpyridine, 1-H-3-methylpyrazol-5-one, 1-phenyl-3-methylpyrazol-5-one, 2,6-dimethyl-pyrazolo[1,5-b]-1,2,4-triazole, 2,6-dimethyl[3,2-c]-1,2,4-triazole, 6-methylpyrazolo[1,5-a]benzimidazole, and their addition salts with an acid.

These couplers preferably represent from 0.0001 to 10% by weight approximately of the total weight of the ready-to-use dyeing composition, and still more preferably from 0.005 to 5% by weight approximately of this weight.

In general, the addition salts with an acid which can be used in the context of the dyeing compositions of the invention (oxidation bases and couplers) are in particular chosen from hydrochlorides, hydrobromides, sulphates and tartrates, lactates and acetates.

The dyeing composition of the invention may also contain, in addition to the oxidation dyes defined above, direct dyes in order to increase the shimmer of the shades. These direct dyes can then in particular be chosen from nitro, azo:or anthraquinone dyes.

The ready-to-use dyeing composition in accordance with the invention may also contain various adjuvants conventionally used in hair dyeing compositions, such as anionic, cationic, nonionic, amphoteric or zwitterionic surfactants or mixtures thereof, polymers, thickeners, antioxidants, enzymes different from the laccases used in accordance with the invention, such as for example peroxidases or oxido-reductases containing 2 electrons, penetrating agents, sequestering agents, perfumes, dispersing agents, film-forming agents, screening agents, vitamins, preservatives or opacifying agents.

Of course, persons skilled in the art will be careful to choose this or these optional additional compounds such that the advantageous properties intrinsically attached to the ready-to-use dyeing composition in accordance with the invention are not, or substantially not, impaired by the addition(s) envisaged.

The ready-to-use dyeing composition in accordance with the invention can be provided in various forms, such as in the form of liquids, creams, gels, optionally pressurized, or in any other form appropriate for dyeing keratinous fibres in particular human hair. In this case, the oxidation dye(s) and the laccase(s) are present in the same ready-to-use composition, and consequently the said composition should be free of gaseous oxygen, so as to avoid any premature oxidation of the oxidation dye(s).

The subject of the invention is also a method of dyeing keratinous fibres, and in particular human keratinous fibres such as hair, using the ready-to-use dyeing composition as defined above.

According to this method, at least one ready-to-use dyeing composition as defined above is applied to the fibres for a sufficient time to develop the desired colour, after which they are rinsed, optionally washed with shampoo, rinsed again and dried.

The time necessary for the development of the colour on the keratinous fibres is generally between 3 and 60 minutes and still more precisely 5 and 40 minutes.

According to one particular embodiment of the invention, the method comprises a preliminary step consisting in storing in a separate form, on the one hand, a composition (A) comprising, in a medium appropriate for dyeing, at least one oxidation dye as defined above and, on the other hand, a composition (B) containing, in a medium appropriate for dyeing, at least one enzyme of the laccase type and at least one particular alkalinizing agent as defined above, and then in mixing them at the time of use before applying this mixture to the keratinous fibres.

According to a specific embodiment of the invention, the alkalinizing agent may be incorporated into the composition (A).

Another subject of the invention is a multi-compartment device or dyeing (kit) or any other multi-compartment packaging system in which a first comparment contains the composition (A) as defined above and a second compartment contains a composition (B) as defined above. These devices may be equipped with a means which makes it possible to deliver the desired mixture to the hair, such as the devices described in Patent FR-2,586,913 in the name of the applicant.

The medium appropriate for keratinous fibres (or carrier) of the dyeing compositions in accordance with the invention generally consists of water or of a mixture of water and of at least one organic solvent in order to solubilize the compounds which might not be sufficiently soluble in water. As organic solvent, there may be mentioned for example C₁-C₄ alkanols such as ethanol and isopropanol as well as aromatic alcohols such as benzyl alcohol, similar products and mixtures thereof.

The solvents may be present in proportions preferably of between 1 and 40% by weight approximately relative to the total weight of the dyeing composition, and still more preferably between 5 and 30% by weight approximately.

The pH of the dyeing compositions in accordance with the invention is chosen such that the enzymatic activity of the laccase is not impaired. It varies generally from 6 to 11 approximately, and more preferably from 6 to 9 approximately.

Concrete examples illustrating the invention will now be given.

In the text which follows and in the preceding text, unless otherwise stated, the percentages are expressed by weight.

The examples below illustrate the invention with no limitation being implied.

### EXAMPLE 1

Dyeing Composition

The following ready-to-use dyeing composition was prepared (contents in grams):

| Laccase obtained from Rhus vernicifera | | 1.8 | g |
|---|---|---|---|
| containing 180 units/mg units/mg, marketed | | | |
| by the company SIGMA | | | |
| (C₈-C₁₀) Alkyl polyglucoside in aqueous | | 8.0 | g |
| solution containing 60% of active | | | |
| substance (AS) sold under the name ORAMIX | | | |
| CG110 by the company SEPPIC | | | |
| Paraphenylenediamine | | 0.254 | g |
| 2,4-Diaminophenoxyethanol dihydrochloride | | 0.260 | g |
| Arginine | qs | pH 6.5 | |
| Demineralized water | qs | 100 | g |

This ready-to-use dyeing composition was applied to locks of natural grey hair which is 90% white for 40 minutes at 30° C. The hair was then rinsed, washed with a standard shampoo and then dried.

Locks of hair with bluish grey colour were obtained.

In this example, 1.8 g of laccase obtained from Rhus vernicifera containing 180 units/mg can be replaced by 1 g of laccase obtained from Pyricularia Orizae containing 100 units/mg sold by the company I.C.N.

### EXAMPLE 2

Dyeing Composition

The following ready-to-use dyeing composition was prepared (contents in grams):

| Laccase obtained from Rhus vernicifera | | 1.8 | g |
|---|---|---|---|
| containing 180 units/mg units/mg, marketed | | | |
| by the company SIGMA | | | |
| (C₈-C₁₀) Alkyl polyglucoside in aqueous | | 8.0 | g |
| solution containing 60% of active | | | |
| substance (AS) sold under the name ORAMIX | | | |
| CG110 by the company SEPPIC | | | |
| Paraphenylenediamine | | 0.254 | g |
| 2,4-Diaminophenoxyethanol dihydrochloride | | 0.260 | g |
| Ethanol | | 20.0 | g |
| Citrulline | qs | pH 8.0 | |
| Demineralized water | qs | 100 | g |

This ready-to-use dyeing composition was applied to locks of natural grey hair which is 90% white for 40 minutes at 30° C. The hair was then rinsed, washed with a standard shampoo and then dried.

Locks of hair with bluish grey colour were obtained.

In this example, 1.8 g of laccase obtained from Rhus vernicifera containing 180 units/mg can be replaced by 1 g of laccase obtained from Pyricularia Orizae containing 100 units/mg sold by the company I.C.N.

1-22. (canceled)

23. A composition for the oxidation dyeing of keratinous fibers comprising:
(a) at least one enzyme of the laccase type;
(b) at least one alkalinizing agent chosen from:
(I) basic amino acids;
(ii) compounds of the following formula (A):
X(OH)₆
wherein n is equal to 1 or 2; X is chosen from K, Li and N⁺R₁R₂R₃R₄ wherein R₁, R₂, R₃, and R₄, which are identical or different, are each chosen from C₁-C₄ alkyl groups, C₁-C₄ monohydroxyalkyl groups and C₂-C₄ polyhydroxyalkyl groups when n=1; or
X is chosen from Mg and Ca when n=2;
(iii) compounds of the following formula (B): 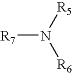
wherein R₅ is chosen from hydrogen groups, C1-C6 alkyl groups, C1-C6 monohydroxyalkyl groups, and C2-C6 polyhydroxyalkyl groups;
R₆ and R₇, which are identical or different, are each chosen from hydrogen groups, C₁-C₆ alkyl groups, C₁-C₆ monohydroxyalkyl groups, and C₂-C₆ polyhydroxyalkyl groups;
with the proviso that R₅, R₆, and R₇ are not simultaneously chosen from C₂β-hydroxyalkyl groups;
with the additional proviso that if R₆ and R₇ are simultaneously chosen from H, then R₅ is not chosen from C₂ monohydroxyalkyl groups and branched C₄ monohydroxyalkyl groups;
and with the additional proviso that if R₅ is chosen from H and C₁-C₆ alkyl groups and simultaneously R₆ is chosen from C₁-C₆ alkyl groups, then R₇ is not chosen from H and C₁-C₆ alkyl groups; and
(iv) compounds of the following formula (C): 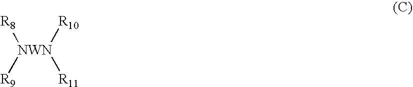
wherein W is chosen from propylene groups optionally substituted with a substituent chosen from hydroxyl groups and C₁-C₄ alkyl groups; R₈, R₉, R₁₀ and R₁₁, which are identical or different, are each chosen from hydrogen groups, C₁-C₄ alkyl groups and C₁-C₄ hydroxyalkyl groups; and
(c) at least one oxidation dye with the proviso that said at least one oxidation dye is not chosen from autooxidizable indole dyes.

24. A composition according to claim 23, wherein said at least one enzyme of the laccase type is chosen from laccases of plant origin, animal origin, fungal origin, and bacterial origin and laccases obtained by biotechnology.

25. A composition according to claim 23, wherein said at least one enzyme of the laccase type is chosen from those produced by plants performing chlorophyll synthesis.

26. A composition according to claim 23, wherein said at least one enzyme of the laccase type is chosen from those extracted from plants chosen from Anacardiaceae, Podocarpaceae, Rosmarinus off., Solanum tuberosum, Iris sp., Coffea sp., Daucus carrota, Vinca minor, Persea americana, Catharenthus roseus, Musa sp., Malus pumila, Gingko biloba, Monotropa hypopithys, Aesculus sp., Acer pseudoplatanus, Prunus persica and Pistacia palaestina.

27. A composition according to claim 23, wherein said at least one enzyme of the laccase type is chosen from those derived from fungi chosen form Pyricularia orizae, Polyporus versicolor, Rhizoctonia praticola, Rhus vernicifera, Scytalidium, Polyporus pinsitus, Myceliophtora thermophila, Rhizoctonia solani, Tramates versicolor, Fomes fomentarius, Chaetomium thermophile, Neurospora crassa, Coriolus versicol, Botrytis cinerea, Rigidoporus lignosus, Phellinus noxius, Pleurotus ostreatus, Aspergillus nidulans, Podospora anserina, Agaricus bisporus, Ganoderma lucidum, Glomerella cingulata, Lactarius piperatus, Russula delica, Heterobasidion annosum, Thelephora terrestris, Cladosporium cladosporioides, Cerrena unicolor, Coriolus hirsutus, Ceriporiopsis subvermispora, Coprinus cinereus, Panaeolus papillionaceus, Panaeolus sphinctrinus, Schizophyllum commune, Dichomitius squalens and variants of all said fungi.

28. A composition according to claim 23, wherein said at least one enzyme of the laccase type is present in a quantity ranging from 0.5 to 2000 lacu units per 100 g of said composition.

29. A composition according to claim 23, wherein said at least one enzyme of the laccase type is present in a quantity ranging from 1000 to 4×10⁷ u units per 100 g of said composition.

30. A composition according to claim 23, wherein said at least one enzyme of the laccase type is present in a quantity ranging from 20 to 2×106 ulac units per 100 g of said composition.

31. A composition according to claim 23, wherein said basic amino acids are chosen from the following formula (D): 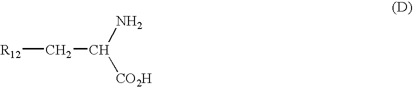
wherein R₁₂ is chosen from:

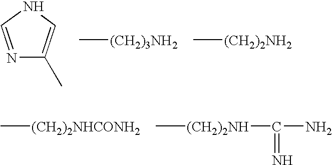

32. A composition according to claim 23, wherein said compounds of formula (B) are chosen from diethanolamine, monoisopropanolamine, diisopropanolamine, triisopropanolamine, 2-amino-2-methyl-1,3-propanediol, 2-amino-2-ethyl-1,3-propanediol, 2-amino-1-n-butanol, 1-diethylamino-2,3-propane-diol, tris(hydroxymethyl)aminomethane and ethylmonoethanolamine.

33. A composition according to claim 23, wherein said at least one alkalinizing agent is present in a quantity ranging from 0.001% to 20% by weight relative to the total weight of said composition.

34. A composition according to claim 33, wherein said at least one alkalinizing agent is present in a quantity ranging from 0.01% to 5% by weight relative to the total weight of said composition.

35. A composition according to claim 34, wherein said at least one alkalinizing agent is present in a quantity ranging from 0.05% to 3% by weight relative to the total weight of said composition.

36. A composition according to claim 23, wherein said at least one oxidation dye is at least one oxidation base chosen from ortho- and paraphenylenediamines, ortho- and para-aminophenols, heterocyclic bases, and the acid addition salts of all said oxidation bases.

37. A composition according to claim 36. wherein said at least one oxidation base is present in a concentration ranging from 0.0005% to 12% by weight relative to the total weight of said composition.

38. A composition according to claim 37, wherein said at least one oxidation base is present in a concentration ranging from 0.005% to 6% by weight relative to the total weight of said composition.

39. A composition according to claim 36, wherein said acid addition salts are chosen from hydrochiorides, hydrobromides, sulphates, tartrates, lactates and acetates.

40. A composition according to claim 23, wherein said at least one oxidation dye is at least one coupler chosen from meta-phenylenediamines, metaaminophenols, meta-diphenols, heterocyclic couplers and the acid addition salts of all said couplers.

41. A composition according to claim 40, wherein said at least one coupler is chosen from 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, sesamol, a-naphthol, 6-hydroxyindole, 4-hydroxyindole, 4-hydroxy-N-methylindole, 6-hydroxyindoline, 2,6-dihydroxy-4-methylpyridine, 1-H-3-methylpyrazol-5-one, 1-phenyl-3-methylpyrazol-5-one, 2,6-dimethylpyrazolo[1,5-b]-1,2,4-triazole, 2,6-dimethyl[3,2-c]-1,2,4-triazole, 6-methylpyrazolo[1,5-a]benzimidazole and the acid addition salts of all said couplers.

42. A composition according to claim 40, wherein said at least one coupler is present in a concentration ranging from 0.0001% to 10% by weight relative to the total weight of said composition.

43. A composition according to claim 42, wherein said at least one coupler is present in a concentration ranging from 0.005% to 5% by weight relative to the total weight of said composition.

44. A composition according to claim 40, wherein said acid salts are chosen from hydrochiorides, hydrobromides, sulphates, tartrates, lactates and acetates.

45. A composition according to claim 23, further comprising at least one direct dye.

46. A composition according to claim 45, wherein said at least one direct dye is chosen from nitro, azo and anthraquinone dyes.

47. A composition according to claim 23, further comprising at least one carrier appropriate for keratinous fibers.

48. A composition according to claim 47, wherein said at least one carrier is chosen from water and at least one organic solvent.

49. A composition according to claim 48, wherein said at least one organic solvent is present in a concentration ranging from 1% to 40% by weight relative to the total weight of said composition.

50. A composition according to claim 49, wherein said at least one organic solvent is present in a concentration ranging from 5% to 30% by weight relative to the total weight of said composition.

51. A composition according to claim 23, having a pH from about 4 to about 11.

52. A composition according to claim 51, wherein said pH varies from about 6 to about 9.

53. A composition according to claim 23, further comprising at least one suitable cosmetic adjuvant chosen from surfactants, polymers, thickeners, antioxidants, enzymes different from said at least one enzyme of the laccase type as defined in claim 23, penetrating agents, sequestering agents, perfumes, dispersing agents, film-forming agents, screening agents, vitamins, preservatives and opacifying agents.

54. A composition according to claim 23 in the form of an aqueous or aqueous/alcoholic lotion, a gel, a milk, a cream, an emulsion, a thickened lotion or a thickened foam.

55. A composition according to claim 23, wherein said composition is ready-to-use.

56. A composition according to claim 23, wherein said keratinous fibers are human keratinous fibers.

57. A composition according to claim 56, wherein said human keratinous fibers are hair.

58. A method of dyeing keratinous fibers comprising applying to said keratinous fibers for a sufficient time to develop a desired color at least one dyeing composition comprising:
(a) at least one enzyme of the laccase type;
(b) at least one alkalinizing agent chosen from:
(i) basic amino acids;
(ii) compounds of the following formula (A):
X(OH)ₙ
wherein n is equal to 1 or 2; X is chosen from K, Li and N⁺R₁R₂R₃R₄ wherein R₁, R₂, R₃, and R₄, which are identical or different, are each chosen from C₁-C₄ alkyl groups, C₁-C₄ monohydroxyalkyl groups and C₂-C₄ polyhydroxyalkyl groups when n1; or
X is chosen from Mg and Ca when n=2;
(iii) compounds of the following formula (B): 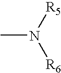
wherein R₅ is chosen from hydrogen groups, C₁-C₆ alkyl groups, C₁-C₆ monohydroxyalkyl groups, and C₂-C₆ polyhydroxyalkyl groups;
R₆ and R₇, which are identical or different, are each chosen from hydrogen groups, C₁-C₆ alkyl groups, C₁-C₆ monohydroxyalkyl groups, and C₂-C₆ polyhydroxyalkyl groups;
with the proviso that R₅, R₆, and R₇ are not simultaneously chosen from C₂β-hydroxyalkyl groups;
with the additional proviso that if R₆ and R₇ are simultaneously chosen from H, then R₅ is not chosen from C₂ monohydroxyalkyl groups and branched C₄ monohydroxyalkyl groups;
and with the additional proviso that if R₅ is chosen from H and C₁-C₆ alkyl groups and simultaneously R₆ is chosen from C₁-C₆ alkyl groups, then R₇ is not chosen from H and C₁-C₆ alkyl groups; and
(iv) compounds of the following formula (C): 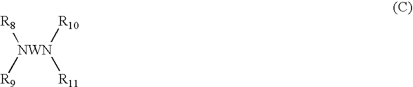
wherein W is chosen from propylene groups optionally substituted with a substituent chosen from hydroxyl groups and C₁-C₄ alkyl groups; R₈, R₉, R₁₀ and R₁₁, which are identical or different, are each chosen from hydrogen groups, C₁-C₄ alkyl groups and C₁-C₄ hydroxyalkyl groups; and
(c) at least one oxidation dye with the proviso that said at least one oxidation dye is not chosen from autooxidizable indole dyes.

59. A method of dyeing keratinous fibers according to claim 58, wherein said keratinous fibers are human keratinous fibers.

60. A method of dyeing keratinous fibers according to claim 59, wherein said human keratinous fibers are hair.

61. A method for dyeing keratinous fibers comprising the steps of:
(a) storing a first composition,
(b) storing a second composition separately from said first composition,
(c) mixing the first composition with the second composition to form a mixture, and
(d) applying said mixture to said keratinous fibers for a time sufficient to achieve a desired coloration,
wherein said first composition comprises said at least one oxidation dye in a medium appropriate for keratinous fibers, and
wherein said second composition comprises said at least one enzyme of the laccase type and said at least one alkalinizing agent in a medium appropriate for keratinous fibers.

62. A multicompartment device or a dyeing kit, comprising a first compartment containing a composition (A) comprising, in a medium appropriate for dyeing, at least one oxidation dye and a second compartment containing a composition (B), comprising, in a medium appropriate for keratinous fibers, at least one enzyme of the laccase type, wherein at least one of said composition (A) and composition (B) comprises at least one alkalinizing agent chosen from:
(i) basic amino acids;
(ii) compounds of the following formula (A):
X(OH)N
wherein n is equal to 1 or 2; X is chosen from K, Li and N⁺R₁R₂R₃R₄ wherein R₁, R₂, R₃, and R₄, which are identical or different, are each chosen from C₁-C₄ alkyl groups, C₁-C₄ monohydroxyalkyl groups and C₂-C₄ polyhydroxyalkyl groups when n=1; or
X is chosen from Mg and Ca when n=2;
(iii) compounds of the following formula (B): 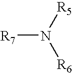
wherein R₅ is chosen from hydrogen groups, C₁-C₆ alkyl groups, C₁-C₆ monohydroxyalkyl groups, and C₂-C₆ polyhydroxyalkyl groups;
R₆ and R₇, which are identical or different, are each chosen from hydrogen groups, C₁-C₆ alkyl groups, C₁-C₆ monohydroxyalkyl groups, and C₂-C₆ polyhydroxyalkyl groups;
with the proviso that R₅, R₆, and R₇ are not simultaneously chosen from C₂β-hydroxyalkyl groups;
with the additional proviso that if R₆ and R₇ are simultaneously chosen from H, then R₅ is not chosen from C₂ monohydroxyalkyl groups and branched C₄ monohydroxyalkyl groups;
and with the additional proviso that if R₅ is chosen from H and C₁-C₆ alkyl groups and simultaneously R₆ is chosen from C₁-C₆ alkyl groups, then R₇ is not chosen from H and C₁-C₆ alkyl groups; and
(iv) compounds of the following formula (C): 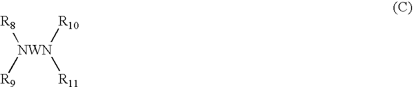
wherein W is chosen from propylene groups optionally substituted with a substituent chosen from hydroxyl groups and C₁-C₄ alkyl groups; R₈, R₉, R₁₀ and R₁₁, which are identical or different, are each chosen from hydrogen groups, C₁-C₄ alkyl groups and C₁-C₄ hydroxyalkyl groups.
